(19) United States
(12) Patent Application Publication
(10) Pub. No.: US 2017/0258710 A1
(43) Pub. Date: Sep. 14, 2017

(54) MONTELUKAST TRANSMUCOSAL FILM

(71) Applicant: Intelgenx Corp., St-Laurent (CA)

(72) Inventors: Justin Conway, Greenfield Park (CA); Rodolphe Obeid, St-Laurent (CA); Nadine Paiement, St-Laurent (CA); Horst Zerbe, Hudson (CA)

(73) Assignee: Intelgenx Corp., St-Laurent (CA)

(21) Appl. No.: 15/067,309

(22) Filed: Mar. 11, 2016

(51) Int. Cl. A61K 9/00; A61K 47/36; A61K 9/70; A61K 31/47

(52) U.S. Cl. CPC A61K 9/006; A61K 31/47; A61K 47/36; A61K 9/7007

(57) ABSTRACT

An oral film product in which a pharmaceutically active agent is stabilized in its partially-ionized form to better facilitate oral transmucosal delivery is provided. The film includes a bioadhesive layer including a pharmaceutically active agent having a logarithmic acid dissociation constant that is less than 4.5 and which is complexed with a cationic polymer.

### CROSS-REFERENCE TO RELATED APPLICATIONS

Not Applicable

### FIELD OF THE DISCLOSURE

This disclosure relates to oral film dosage forms for transmucosal delivery of a pharmaceutically active agent, and more specifically to such dosage forms for transmucosal delivery of a pharmaceutically active agent that is resistant to transmucosal absorption due to a low acid dissociation constant.

### BACKGROUND OF THE DISCLOSURE

It is often beneficial to administer pharmaceutically active compounds via mucosal tissue. In some cases, transmucosal delivery eliminates first-pass or presystemic metabolism, often very substantially increasing bioavailability, reducing the dosing needed for a desired therapeutic effect (and thereby reducing cost of the treatment), and reducing individual variation in the required safe and effective dose due to differences in the extent of first-pass metabolism. In some cases, transmucosal delivery is desirable for rapid onset of therapeutic effect.

Despite the many advantages of transmucosal drug delivery, not all pharmaceutically active agents can be easily delivered through mucosal tissue. For example, certain ionizable pharmaceutically active agents having a low acid dissociation constant are resistant to absorption via oral mucosal tissue due to electrostatic repulsion. This problem can be illustrated by reference to the active agent Montelukast.

Montelukast is leukotriene receptor antagonist used to treat asthma and relieve symptoms associated with seasonal allergies. It is generally available in a tablet form which has been shown to have limited bioavailability due to first-pass hepatic metabolism. Montelukast bioavailability is around 64% but the absorption is known to be variable and is impacted by food consumption at lower dose strengths.

It would be desirable to address this limitation through the development of a Montelukast film with enhanced bioavailability by exploiting the transmucosal absorption pathway in the buccal cavity. Buccal absorption would limit the API (active pharmaceutical ingredient) metabolization and ensure an increased amount of drug reaches the blood stream. Buccal adsorption will also reduce the food sensitivity observed with the lower strength dose of Montelukast. Fast onset of action is not the primary objective in this case but could be a result as the time to reach maximum concentration (3 to 4 hours) is fairly long.

There is a technical challenge associated with the absorption of Montelukast in the buccal cavity because of the low pKa of 4, which would generate a negative charge on the molecule in a saliva buffer environment. Since mucins, the main constituent in mucosa, are also negatively charged, electrostatic repulsive forces between the mucosal surface and the negatively charged carboxyl groups of Montelukast will impede permeability through the membrane.

As a matter of fact, various drugs can only be absorbed through the buccal mucosa if they are in between their low- and non-ionized states, defined as partially-ionized form. Taking into consideration the pKa of Montelukast, the pH for a Montelukast containing film must be adjusted to a value near or below its pKa to reduce the charge density or prevent the full ionization state. However, simply acidifying the film formulation to stabilize the partially ionized form of Montelukast may lead to oral irritation when administered to patients. Furthermore, Montelukast solubility is highly dependent on pH, exhibiting good solubility at a pH over 7 and quickly precipitating under slightly acidic conditions. The pH within a human mouth is between 5.8 and 7.4. A novel Montelukast film formulation must therefore attain a balance between film pH, API solubility, and efficient absorption in order to penetrate the mucosa into the blood stream.

### SUMMARY OF THE DISCLOSURE

Disclosed is an oral film product in which a pharmaceutically active agent is stabilized in its partially-ionized form through the use of an acidic formula matrix with or without the use of surfactant. This partially-ionized form of API shows unexpected good permeability owing to the incorporation of muco-adhesive polymers within the formula. An optional protective backing layer can be used to further increase the local concentration at the membrane interface and minimize ionization of the active agent due to the local pH environment within the oral cavity.

These and other features, advantages and objects of the various embodiments will be better understood with reference to the following specification and claims.

### DETAILED DESCRIPTION

The oral transmucosal delivery devices disclosed herein have a bioadhesive layer that comprises, consists essentially of, or consists of, a pharmaceutically active agent having a logarithmic acid dissociation constant (pKa) that is less than 4.5 and that is interacted with a cationic polymer. By complexing the low pKa active agent with a cationic polymer, the resulting complex can be optionally combined with other polymers, surfactants, adjuvants, and/or excipients, and cast into a bioadhesive film in which the active agent is stabilized in its partially-ionized form. By incorporating the low-pKa active agent into a bioadhesive film layer in a partially-ionized form, unexpectedly improved permeability of the active agent into oral mucosal tissue can be achieved.

Examples of pharmaceutically active agents that have a low pKa (i.e., less than about 4) include amidinocillin (3.4), aminohippuric acid (3.8), amoxicillin (2.4), ampicillin (2.5), azlocillin (2.8), aztrenam (0.7), carbenicillin (2.7), cefaclor (1.5), cefamandole (2.7), cefazolin (2.1), cefoperazone (2.6), cefotaxime (3.4), cefoxitin (2.2) ceftazidime (1.8), ceftizoxime (2.7), ceftriazone (3.2), cephalexin (3.2), cephaloridine (3.4), cephalothin (2.5), chlortetracycline (3.3), clofibrate (3.5), cloxacillin (2.8), cromolyn (1.1), cyclacillan (2.7), demeclocycline (3.3), diatrizoic acid (3.4), dicloxacillin (2.8), diflunisal (3), doxycycline (3.4), enalaprilat (2.3), erialapril (3), ethacrynic acid (3.5), flucloxacillin (2.7), flufenamic acid (3.9), furosemide (3.9), hippuric acid (3.6), iodipamide (3.5), leucovorin (3.1), levodopa (2.3), levothyroxine (2.2), lisinopril (1.7), mercaptomerin (3.7), mesalamine (2.7), methacycline (3.5), methicillin (3), methotrexate (3.8), methyldopa (2.3), metyrosine (2.7), mezlocillin (2.7), minocycline (2.8), moxalactam (2.5), nafcillin (2.7), niacin (2), oxacillin (2.7), oxytetracycline (3.3), p-aminosalicyclic acid (3.6), penicillamine (1.8), penicillin G (2.8), penicillin V (2.7), phenethicillin (2.8), probenecid (3.4), rifampin (1.7), salicyclic acid (3), salsalate (3.5), sulfasalazine (2.4), sulfinpyrazone (2.8), tetracycline (3.3), ticarcillin (2.6), ticrynafen (2.7), tiprofenic acid (3), tolazamide (3.1), and tolmetin (3.5).

An example of a low-pKa pharmaceutically active agent that can be complexed with a cationic polymer incorporated into a bioadhesive film layer useful for achieving enhanced oral transmucosal delivery is Montelukast (pKa=4.4). However, the disclosed technique of stabilizing a low-pKa active agent with a cationic polymer to facilitate or enhance permeation via oral mucosal tissue can be applied to numerous other active agents that might otherwise be resistant to transmucosal delivery.

The cationic polymer can be any pharmaceutically acceptable polymer capable of complexing with the active agent, and exhibiting mucoadhesivity in the oral cavity of a subject, and/or compatible and combinable with oral mucoadhesive materials to facilitate adhesion to oral mucosal tissue (e.g., buccal and labial mucosa). Examples of cationic polysaccharide polymers that exhibit bioadhesion in the oral cavity include cationic chitosan, cationic poly(amino acids), cationic dextran, cationic cellulose, and cationic cyclodextrin and/or their copolymer analogs. Such materials are commercially available, and/or have been thoroughly described in the open literature. Other cationic polymers and copolymers that may be used to prepare the active agent—cationic polymer complex include polyethylene imine, poly-L-lysine, poly(amidoamine)s, poly (amino-co-ester)s, and poly(2-N—N-dimethylaminoethylmethacrylate), and their copolymer analogs, all of which are thoroughly described in the open literature.

If necessary or desirable, the active agent—cationic polymer complex can be combined or blended with other film forming polymers and/or mucoadhesive polymers to obtain a balanced combination of properties suitable for an oral transmucosal delivery device. Examples of suitable film forming polymers exhibiting mucoadhesion include hydroxypropyl cellulose, hydroxymethylcellulose, natural or synthetic gum, polyvinyl alcohol, polyethylene oxide, homo- and copolymers of acrylic acid crosslinked with a polyalkenyl polyether or divinyl alcohol, polyvinylpyrrolidone, hydroxypropylmethyl cellulose, sodium alginate, pectin, gelatin and maltodextrins. In certain embodiments or aspects of this disclosure, the active agent—cationic polymer complexes are combined with film forming neutral polysaccharides such as pullulan.

In order to further inhibit ionization of the active agent after administration (i.e., application to oral mucosa) and during transmucosal delivery of the active agent, the bioadhesive film can further comprise an acidifying agent in an amount that is sufficient to adjust the local pH in the bioadhesive layer, after it has been adhered to oral mucosa and imbibed with saliva, to a value of from about 6 to about 3. Acidifying agents that are pharmaceutically acceptable include 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, malcic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid.

Buffers may be employed as needed or as desired to maintain a desirable pH.

Although complexing with a cationic polymer greatly enhances permeability of low-pKa active agent through oral mucosa, penetration enhancing agents can be employed to further increase the rate and/or total amount of absorption of the active agent. Examples of penetration enhancers that can be advantageously employed include 2,3-lauryl ether, phosphatidylcholine, aprotinin, polyoxyethylene, azone, polysorbate 80, benzalkonium chloride, polyoxyethylene, cetylpyridinium chloride, phosphatidylcholine, cetyltrimethyl ammonium bromide, sodium EDTA, cyclodextrin, sodium glycocholate, dextran sulfate 16 sodium glycodeoxycholate. Other penetration enhancers include surfactants, bile salts (by extracting membrane protein or lipids, by membrane fluidization, by producing reverse micellization in the membrane and creating aqueous channels), fatty acids (that act by disrupting intercellular lipid packing), azone (by creating a region of fluidity in intercellular lipids) and alcohols (by reorganizing the lipid domains and by changing protein conformation). Examples of surfactants that can be employed to enhance penetration and/or wettability of the film to promote adhesion include polysorbates (Tween™), sodium dodecyl sulfate (sodium lauryl sulfate), lauryl dimethyl amine oxide, cetyltrimethylammonium bromide (CTAB), polyethoxylated alcohols, polyoxyethylene sorbitan octoxynol (Triton X 100™), N,N-dimethyldodecylamine-N-oxide, hexadecyltrimethylammonium bromide (HTAB), polyoxyl 10 lauryl ether, Brij 721™, bile salts (sodium deoxycholate, sodium cholate) polyoxyl castor oil (Cremophor™), nonylphenol ethoxylate (Tergitol™), cyclodextrins, lecithin, methylbenzethonium chloride (Hyamine™).

Stability enhancing agents can be added to the bioadhesive film to prevent photodegradation, oxidation, and/or microbial contamination. Photodegradation inhibitors include ultraviolet radiation absorbers and pigments. Ultraviolet absorbers include hydroxyl benzophenones and hydroxyphenyl benzotriazoles. Pigments that can be added to the bioadhesive film include various metal oxides, such as titanium dioxide (TiO₂), ferric oxide (Fe₂O₃), iron oxide (Fe₃O₄), and zinc oxide (ZnO).

Other additives, such as excipients or adjuvants, that can be incorporated into the bioadhesive film include flavors, sweeteners, coloring agents (e.g., dyes), plasticizers, and other conventional additives that do not deleteriously affect transmucosal delivery of the active agent, oral mucoadhesivity, or their important film properties.

The bioadhesive film can be used in a monolayer form, or in a multilayer laminated form. In particular, a barrier layer can be advantageously employed to prevent the active agent from diffusing through the bioadhesive film into the oral cavity of a subject after it is adhered to the subject's oral mucosa, and to prevent the loss of acidifying agents when they are used. The barrier layer is preferably comprised of polymers having a low solubility in water. A combination of water-insoluble polymer(s) and a minor amount of a water-soluble polymer(s) can be employed to maintain a barrier that prevents loss of the active agent to the oral cavity until an effective or desired amount of the active agent has been transmucosally delivered, and which allows erosion and/or dissolution thereafter. In some cases it may be advantageous to employ higher molecular weight polymer analogs of the polymer(s) used in the bioadhesive layer. The higher molecular weight (or, equivalently, higher viscosity) analogs are typically more resistant to diffusion and dissolution, and exhibit better compatibility than if polymers of a different chemical type are used.

Examples of water-insoluble polymers that can be employed in the barrier layer include polysiloxanes (silicone polymers), ethyl cellulose, propyl cellulose, polyethylene, and polypropylene. One or more of these polymers may comprise a majority of the barrier film layer by weight (i.e., at least 50 percent). Water soluble hydroxypropyl cellulose can be used in a minor amount to facilitate erosion and/or dissolution of the barrier layer after it has served its function during transmucosal delivery of the active agent. High viscosity polymer could also be used to create a barrier and limit erosion. For example, hydroxypropyl cellulose, polyethylene oxide, polyvinyl pyrrolidone and any other polymer soluble in water, but exhibiting high viscosity, can be used.

The various examples provided are illustrated, and not limiting.

Intelgenx MTL1:

| Compound | % Dry Mass | % Wet Mass |
|---|---|---|
| Water | — | 76.91 |
| Acacia gum | 8.18 | 1.89 |
| Pullulan | 37.92 | 8.75 |
| Citric acid | 7.98 | 1.84 |
| Dextran | 2.14 | 0.53 |
| Sorbitol | 8.28 | 1.92 |
| Sucralose | 1.01 | 0.23 |
| Glycerol | 8.55 | 1.94 |
| Montelukast | 14.97 | 3.45 |
| Propylylparabene | 0.99 | 0.23 |
| Sodium lauryl sulfate | 9.98 | 2.31 |
| Total | 100 | 100 |

Intelgenx MTL2:

| Compound | % Dry Mass | % Wet Mass |
|---|---|---|
| Water | — | 86.65 |
| Montelukast | 6.93 | 0.93 |
| HPMC | 25.95 | 3.46 |
| Sodium starch glycolate | 1.73 | 0.23 |
| Citric acid | 1.73 | 0.23 |
| PEG | 48.44 | 6.47 |
| Sucralose | 1.73 | 0.23 |
| Cyclodextrin | 4.84 | 0.64 |
| Sodium edetate | 8.65 | 1.16 |
| Total | 100 | 100 |

Intelgenx MTL3:

| Compound | % Dry Mass | % Wet Mass |
|---|---|---|
| Water | — | 92.24 |
| Montelukast | 23.78 | 1.84 |
| Carbopol | 9.51 | 0.74 |
| NaCMC | 32.92 | 2.55 |
| Propylene glycol | 14.27 | 1.10 |
| Glycerol monostearate | 5.23 | 0.42 |
| Sodium edetate | 9.54 | 0.74 |
| Sucralose | 4.75 | 0.37 |
| Total | 100 | 100 |

Intelgenx MTL4:

| Compound | % Dry Mass | % Wet Mass |
|---|---|---|
| Water | — | 78.46 |
| Chitosan LMW | 8.95 | 1.93 |
| HPC MMW | 41.48 | 8.93 |
| Citric acid | 9.30 | 2.00 |
| Xanthan gum | 2.51 | 0.54 |
| PEG | 0.76 | 0.16 |
| Sorbitol | 9.06 | 1.95 |
| Sucralose | 1.09 | 0.24 |
| Glycerol | 9.17 | 1.98 |
| Montelukast | 16.38 | 3.07 |
| Propylylparabene | 1.09 | 0.24 |
| BHT | 0.21 | 0.5 |
| Total | 100 | 100 |

Intelgenx MTL5:

| Compound | % Dry Mass | % Wet Mass |
|---|---|---|
| Water | — | 78.46 |
| Starch | 8.95 | 1.93 |
| HPC MMW | 41.48 | 8.93 |
| Citric acid | 9.30 | 2.00 |
| Chitosan | 2.51 | 0.54 |
| PEG | 0.76 | 0.16 |
| Sorbitol | 9.06 | 1.95 |
| Sucralose | 1.09 | 0.24 |
| Glycerol | 9.17 | 1.98 |
| Montelukast | 16.38 | 3.07 |
| Propylylparabene | 1.09 | 0.24 |
| BHT | 0.21 | 0.5 |
| Total | 100 | 100 |

Intelgenx MTL6:

| Compound | % Dry Mass | % Wet Mass |
|---|---|---|
| Methyl ethyl ketone | — | 59.96 |
| 2-Iso propanol | — | 14.06 |
| Montelukast | 16.75 | 4.34 |
| Sucralose | 0.69 | 0.18 |
| Menthol | 7.37 | 1.91 |
| Triacetin | 4.53 | 1.18 |
| Eudragit | 24.57 | 6.38 |
| Copovidone | 3.48 | 0.91 |
| HPC MMW | 41.54 | 10.78 |
| Titanium dioxide | 1.04 | 0.298 |
| BHT | 0.1 | 0.002 |
| Total | 100 | 100 |

Intelgenx MTL7:

| Compound | % Dry Mass | % Wet Mass |
|---|---|---|
| Methyl ethyl ketone | — | 63.98 |
| 2-Iso propanol | — | 15.01 |
| Montelukast | 22.10 | 4.64 |
| Sucralose | 0.91 | 0.19 |
| Menthol | 9.72 | 2.04 |
| Triacetin | 5.98 | 1.25 |
| Magnasweet | 0.47 | 0.128 |
| Copovidone | 4.60 | 0.96 |
| HPC MMW | 28.57 | 6.51 |
| Titanium dioxide | 1.49 | 0.29 |
| Cyclodextrin | 23.81 | 5.00 |
| BHT | 0.01 | 0.002 |
| Total | 100 | 100 |

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. An oral transmucosal delivery device, comprising:
a bioadhesive layer including a pharmaceutically active agent having a logarithmic acid dissociation constant (pKa) less than 4.5 complexed with a cationic polymer.

2. The device of claim 1, further comprising an acidifying agent in an amount sufficient to adjust the local pH to a value of from about 6 to about 3.

3. The device of claim 1, further comprising a neutral film forming polymer.

4. The device of claim 1, further comprising a film forming neutral polysaccharide.

5. The device of claim 1, further comprising Pullulan.

6. The device of claim 1, further comprising a gum.

7. The device of claim 1, further comprising Acacia gum.

8. The device of claim 1, further comprising a permeation enhancer.

9. The device of claim 8, wherein the permeation enhancer is selected from the group consisting of surfactants, bile salts, fatty acids, laurocapram, and alcohols.

10. The device of claim 1, in which the cationic polymer is a polysaccharide.

11. The device of claim 10, in which the polysaccharide is selected from the group consisting of cationic chitosan, cationic dextran, cationic cellulose, and cationic cyclodextrin.

12. The device of claim 1, in which the cationic polymer is a poly(amino acid).

13. The device of claim 12, in which the poly(amino acid) is selected from the group consisting of poly(lysine), copoly(lysine) and their derivatives alone or in combination with other polymers.

14. The device of claim 1, in which the cationic polymer is selected from the group consisting of polyethylenimine, poly(amidoamine)s, poly(amino-co-ester)s, and poly(2-N—N-dimethylaminoethyl-methacrylate).

15. The device of claim 1, further comprising a stability enhancer.

16. The device of claim 15, wherein the stability enhancer is selected from the group consisting of photodegradation inhibitors, antioxidants, chelating agents, and antimicrobial agents.

17. The device of claim 15, wherein the stability enhancer is a photodegradation inhibitor selected from the group consisting of ultraviolet absorbers and pigments.

18. The device of claim 17, in which the stability enhancer is an ultraviolet absorber selected from the group consisting of hydroxyl benzophenones and hydroxyphenyl benzotriazoles.

19. The device of claim 17, wherein the stability enhancer is a pigment selected from the group consisting of titanium dioxide, ferric oxide, iron oxide and zinc oxide.

20. The device of claim 1, further comprising a barrier layer laminated to the film layer.

21. The device of claim 20, in which the barrier layer comprises a polymer matrix that prevents diffusion of the pharmaceutically active agent from the film layer to an oral cavity of a subject that has been administered the device.

22. The device of claim 21, in which the polymer matrix comprises at least 50 percent by weight of at least one polymer selected from the group consisting of polysiloxanes, ethyl cellulose, propyl cellulose, polyethylene, and polypropylene, and an amount of hydroxypropyl cellulose, polyethylene oxide, and polyvinyl pyrrolidone that is effective to facilitate erosion or dissolution.

23. The device of claim 1, in which the pharmaceutically active agent is a leukotriene receptor antagonist.

24. The device of claim 1, in which the pharmaceutically active agent is Montelukast.
